(19) United States
(12) Patent Application Publication
(10) Pub. No.: US 2015/0219619 A1
(43) Pub. Date: Aug. 6, 2015

(54) METHODS OF AND ASSAY PRODUCTS FOR, DETERMINING THE PREDISPOSITION OF A SUBJECT TO DISEASE STATES CHARACTERISED BY FREE RADICAL INDUCED DNA DAMAGE

(71) Applicant: UNIVERSITY OF BARDFORD, West Yorkshire, Yorkshire (GB)

(72) Inventors: Diana Anderson, Bierley (GB); Mojgan Najafzadeh, Huddersfield (GB); Morgan Clive Thomas Denyer, Bradford (GB)

(73) Assignee: University of Bradford, Bradford (GB)

(21) Appl. No.: 14/426,444

(22) PCT Filed: Sep. 10, 2013
(86) PCT No.: PCT/GB2013/052365

§ 371 Date: Mar. 6, 2015

(30) Foreign Application Priority Data
Sep. 11, 2012 (GB) 1216174.1

(51) Int. Cl. G01N 33/49

(52) U.S. Cl. CPC G01N 33/49; G01N 2800/7009; G01N 2800/7028; G01N 2800/50

(57) ABSTRACT

The present invention relates to a method of determining the predisposition of a subject to cancer or other disease states characterised by free radical induced DNA damage such as COPD or asthma and identifying those with undiagnosed cancer. The method is able to utilise whole blood from a subject and is based on exposing the samples to different levels or intensities of electromagnetic radiation, preferably by altering the distance between the source of radiation and the sample.

The present invention relates to a method of determining free radical induced DNA damage. More particularly to a method of determining the predisposition of a subject to cancer or other disease states characterised by free radical induced DNA damage such as COPD or asthma.

The invention also relates to a method of determining the current status of the subject in relation to certain cancers.

The invention also relates to a method of determining the sensitivity of a subject to electromagnetic (EM) radiation induced cellular damage. In particular, the invention relates to a method of determining the sensitivity of a subject to ultraviolet radiation (UV) induced cellular damage.

The invention also relates to an assay product or interrelated kit for use in performing the methods of the invention.

Oxidative DNA damage to cells is an unavoidable consequence of cellular metabolism; however, interactions with exogenous sources such as carcinogenic compounds, redox-cycling drugs, ionising and UV radiation also contribute to oxidative damage.

UV can be used as a physical generic mutagen to induce DNA damage in vitro. Compared to a chemical genotoxin, the advantage of using UVA light is the exact setting of exposure time. Chemical genotoxins require removal by centrifuging and washing procedures.

UVA is part of the sunlight with its electromagnetic spectrum at sea level (290-5000 nm) not only including the visible (56%) and infrared (39%) part but also ultraviolet (UV) light (5%) Mostly UVA (320-400 nm) and to a lesser extent due to atmospheric absorption UVB (290-320 nm) reaches the earth's surface, while the germicide UVC part is completely filtered off. The UVA/B light has been commonly characterised as an environmental human carcinogen being also responsible for erythema (sun-burn), tanning, photo-aging and immune-suppression. However, artificial UV sources, mainly emitting UVA, can also be found in tanning studios and for the treatment of psoriasis. Absorption of UVA in tissue results in the generation of reactive oxygen and nitrogen species and labile iron which can in turn damage other bio-molecules such as DNA. The most frequent type of DNA damage after UV exposure are cyclobutane pyrimidine dimers (CPD) being mostly contributed by marginal amounts of UVB while UVA induced oxidative DNA damage like 8-oxo 10 guanine depends on non-DNA chromophores present in the cells.

UV absorption generates oxygen derived free radicals inducing DNA damage via the production of a range of photoproducts. This process can change the base pairing abilities of normal DNA resulting in mutations. It is these mutations that may lead to cancers because they disrupt tumour suppressor genes such as P53, and INK4A. Free radicals play a role in numerous types of cancers. There is evidence that free radical related mutations of the ATM (Ataxia-telangiectasia mutated) and ATR (Rad3-related) genes play a role in lymphomagenesis.

Over the past two decades or so, the Comet assay, or single-cell gel electrophoresis assay has become one of the standard methods for evaluating DNA damage by assessing DNA strand breaks in cells. Cells embedded in agarose on a microscope slide are lysed with detergent and high salt concentration to form nucleoids containing supercoiled loops of DNA linked to the nuclear matrix.

Electrophoresis at high pH results in structures resembling comets, observed by fluorescence microscopy in which the intensity of the comet tail relative to the head reflects the number of DNA strand breaks and alkali labile sites. Loops containing a break lose their supercoiling and freely move towards the anode. Thus the Comet assay provides a means of determining genomic damage.

Calculation of fluorescence to determine the extent of DNA damage can be performed by manual scoring using imaging software.

It has been recently found that cancer itself and most likely its pre-cancerous states can cause stress to the entire organism. This fact may then lead to differences in sensitivity to exogenous genotoxic insults in cells unrelated to the cancer. Insults due to environmental stressors or life style factors may subsequently lead to higher damage in sensitive cells.

Previously, the inventors have used traditional comet tests and novel variations thereof (for example see WO2008/050134) as a model for skin to determine the ability of a test substance, such as a sun screen, to protect cells from UV damage. However, this methodology requires significant pre-treatment steps as needed the sample to be in the form of lymphocytes. Furthermore, the lymphocyte sample layer is overlaid with one (for a traditional comet assay) or more (for the novel 3D assay) additional barrier layers to provide a sandwich effect which holds the sample inside. This results in a fairly laborious process.

It would be desirable to provide an improved method of determining the sensitivity of cells to electromagnetic radiation. It would be desirable to determine whether a subject has a predisposition to cancer or other diseases characterised by DNA damage.

According to one aspect of the present invention there is provided a method of screening subjects for a predisposition to diseases characterised by DNA damage, comprising the steps:
obtaining whole blood from a subject;
mounting a plurality of samples of said whole blood on a substrate;
exposing the samples to different levels or intensities of electromagnetic radiation;
detecting the level of genetic damage in each sample; and
comparing the levels of damage with predetermined values, or patterns of values

Preferably the method of screening includes a final step of determining from said comparison the relevant diagnostic result.

The present invention shows a clearer separation between groups than prior art methods. It also allows greater reproducibility of results.

Preferably the step of exposing each sample to a different level of electromagnetic radiation is carried out by altering the distance between the source of radiation and the sample to produce the correct intensity for each sample.

The plurality of samples may be taken as a single sample from a subject provided that it can be separated into a plurality of samples for testing.

Preferably samples are exposed to electromagnetic radiation for between 10 minutes and 30 minutes

Most preferably the samples are exposed to electromagnetic radiation 15 minutes.

Optionally, the step exposing the samples to varying levels of electromagnetic radiation is carried out varying the levels of electromagnetic radiation emitted by adjusting the height.

Most preferably, no additional layer of barrier material is placed between the samples and the source of electromagnetic radiation.

Most preferably, no additional layer of barrier material is placed directly on top of the samples.

Preferably, the whole blood comprises peripheral whole blood.

It is particularly advantageous to be able to use whole blood without needing significant pre-treatment steps.

Preferably, before exposing samples to different levels of electromagnetic radiation, the method includes the step of:
mounting the whole blood on a substrate.

In order to mount the whole blood samples onto a substrate, typically 40 μl of diluted whole blood is mixed with an equal amount of cell culture medium (RPMI) and 10% of DMSO (storage and freezing medium), and this is mixed with 100 μl of 0.5% low melting point agarose (<40° C.) and applied to dry agarose-coated slides.

It is envisaged that 35 μl, 40 μl or 45 μl of blood would be preferred volumes. Cell numbers in these cases would be 7-10, 10-15 or 20-25 cells respectively according to blood volume. Density of cells would be 0.0015, 0.0020 or 0.0030 respectively according to blood volume.

Most preferably, the mounting medium is 0.5% low-melting point agarose 40° C.).

Preferably the EM radiation is UV radiation.

As the sample is provided on a substrate without any additional barrier layer being provided on top, it is possible to lyse the whole blood cells after they have been exposed to EM radiation, thus removing the need for laborious pre-treatment steps.

After lysis, the method may also include the step of:
removing the lysing solution containing red blood cells and debris.

The method could be used to screen subjects for a predisposition to cancers including but not limited to skin cancer, lung cancer, breast cancer, bowel cancer, prostate cancer, colo-rectal cancer.

The method could also be used to screen subjects for a predisposition to COPD, asthma, emphysema or polyposis coli.

In order to provide a better understanding of the present invention, embodiments will now be described with reference to the following figures in which;

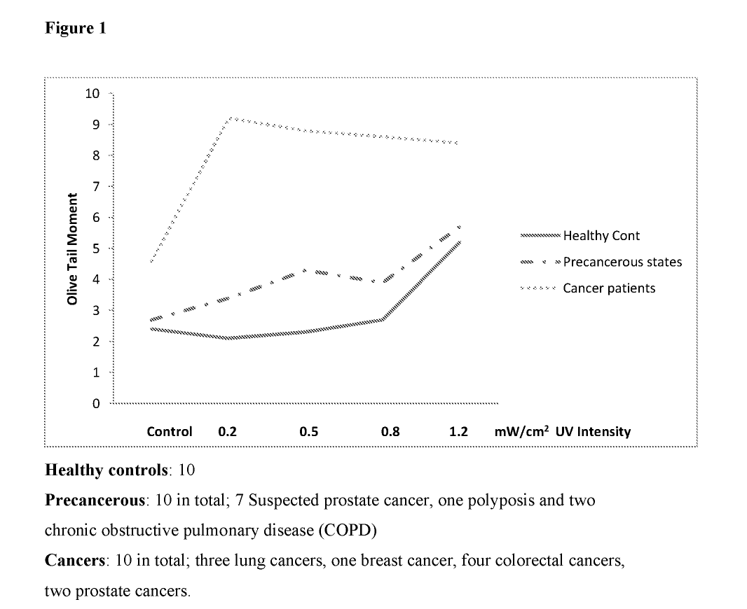

FIG. 1 is a graphical representation of data obtained from examples using the method of the invention, showing olive tail moments of cells at varying height of UV light (i.e. varying distances between sample and UV source, resulting in varying intensities) and showing the difference in profiles between healthy controls, precancerous states and cancers.

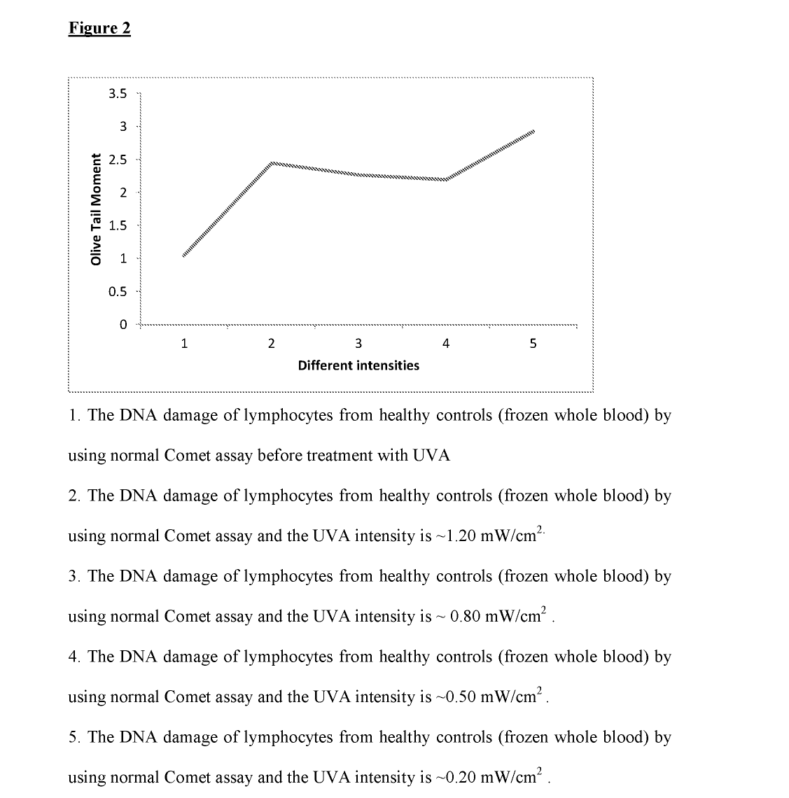

FIG. 2 is a graphical representation of data obtained utilising a standard normal (conventional) Comet Assay protocol as published in the literature, using variable heights of UV light. Notably, the profile here differs from profiles obtained using the new methodology.

Differential responses of cells to UV, a physical generic mutagen, have been found by the inventors to provide an accurate indication of a subject's predisposition to cancer. The present invention provides a simple and accurate method for testing a sample for sensitivity of cells in the sample to UV and using the results to provide an accurate indication of a subject's predisposition to cancer.

The first step is to obtain a sample or samples from a patient. For example, peripheral whole blood samples can be easily collected from a subject by venepuncture. The samples (which can be derived from a single blood sample taken from a subject which has been divided appropriately) can the be used in the following assaying method (generally using an assay product or interrelated kit).

### Preferred Protocol (Based on UVA, Whole Blood and Modified Standard Comet Assay Methodology, I.E. the New Assay)

Glass slides from BDH (Superfrost™) to be used are coated with 1% agarose (Normal Melting Point agarose). All other chemical reagents are obtained from VWR International laboratories suppliers address in the UK. 40 μl of the diluted whole blood with equal amount of RPMI and 10% of DMSO (storage and freezing medium) are mixed with 100 μl of 0.5% low-melting point agarose (<40° C.) and applied to dry agarose-coated slides.

No second layer of LMP agarose is needed as opposed to the three layers forming “a sandwich” in the standard Comet assay. The slides are just left on ice for 5 minutes then they are ready for the UVA treatment.

For UVA exposure, a table-top lamp housing two 15 W PUVA tubes (Waldmann,Villingen—Schwenningen, Germany; bought from Athrodax Healthcare International Ltd, UK) is used. The spectrum of the PUVA tube ranges from 320-410 nm with a maximum at 351 nm. For UV treated slides the lamp is positioned at different distances from the slides to create suitable intensities. The first slide in the treated group is the positive control and the intensity is ˜1.20 mW/cm² and the rest of the slides are at different intensities. The second is ˜0.80 mW/cm², the third is ˜0.50 mW/cm² and the fourth is ˜0.20 mW/cm².

For this new assay, cells are exposed embedded in 0.5% LMP agarose on slides.

There are two series of slides:

### Group 1:

Slide number one is covered with 100 μl of LMP 0.5% agarose without UVA treatment (Negative control).

### Group 2:

Slide number two, is covered with 100 μl of LMP 0.5% agarose and the UVA intensity is ˜1.20 mW/cm² (distance from UVA lamp is 15 cm) (positive control). Slide number three, is covered with 100 μl of LMP 0.5% agarose and the UVA intensity is ˜0.80 mW/cm².(distance from UVA lamp is 20 cm).

Slide number four is covered with 100 μl of LMP 0.5% agarose and the UVA intensity is ˜0.50 mW/cm². (distance from UVA lamp is 25 cm). Slide number five is covered with 100 μl of LMP 0.5% agarose and the UVA intensity is ˜0.20 mW/cm².

The slides are immersed after treatment laterally in a container with cold lysing solution (2.5 M NaCl, 100 mM EDTA, 10 mM Tris, 10% DMSO, 1% Triton X-100, pH 10). The slides are incubated at 4° C. for 1 hour or overnight.

After lysis, the slides are placed horizontally on the tray of an electrophoresis tank, filled with cold electrophoresis buffer (300 mM NaOH, 1 mM EDTA, pH <13) The slides are incubated for 30 minutes at 4° C. in the dark to allow the unwinding of DNA and expression of the single strand breaks (SSB) or double strand breaks (DSB) alkali labile damage. Electrophoresis is conducted at the same temperature for 30 minutes at 25 volts and an adjusted current of 300 mA (by raising or lowering the buffer level).

After electrophoresis, the slides are removed from the tank and soaked three times each with neutralising buffer (400 mM Tris, pH 7.5) for a period of five minutes (Tice, Agurell et al. 2000; Anderson, Schmid et al. 2003).

Cells are stained with 20 μg/ml ethidium bromide or an appropriate alternative e.g. SYBR Green and examined using a fluorescence microscope equipped with a monochrome CCD-camera.

For each replicate slide, 50 cells are scored (100 cells in total, making 1000 observations per experimental point allowing a more than adequate statistical power to detect effects).

A computerised image analysis system, Komet 6.0 (Andor Ltd, Belfast), is employed to measure the Comet parameters; the median Olive tail moment and % tail DNA are then used for statistical analysis.

The results of an experiment carried out on samples obtained from 30 subjects, including those termed pre cancerous, those termed cancerous and healthy controls, using the protocol can be seen in FIG. 1.

Olive tail moment is indicative of DNA damage and thus one would expect that in all cells, olive tail moment of cells would increase with increased intensity of exposure to the UV mutagen (i.e. with decreasing height of the UV lamp). This is clearly the case in FIG. 1, using the method of the invention. In contrast, using a standard normal (conventional) sample Comet assay where an additional barrier layer is provided above the sample, and varying the lamp height, the results are not consistent with this principle (FIG. 2).

Further, FIG. 1 shows that cells of an individual having cancerous or precancerous conditions follow a similar profile, which is very distinct from that observed for cells of healthy individuals. The healthy controls show a very different profile to either cancerous or pre-cancerous cells. This allows clear identification of the subjects whose samples indicate a predisposition to cancer. The profile shown in FIG. 2, using standard normal (conventional) comet assay could not be used in this manner as it is not sufficiently distinguishable from healthy controls to provide a clear and accurate result.

It is notable that if using a conventional comet assay (the results of which are shown in FIG. 2) there are some significant technical difficulties and problems to prepare the slides from whole blood and treatment with the UVA.
1. Mixing the whole blood with 1% LMP agarose and spreading it on the slide, it is not easily spread on the slide.
2. The second layer of agar 100 μl of 0.5% LMP cannot cover the entire slide at the time of applying.
3. After using lysing solution it is not possible to wash out all of the red blood cells that have been attached to the agar gel.

The present invention aims to mitigate these problems.

1. A method of screening subjects for a predisposition to or presence of one or more diseases characterized by DNA damage the method comprising:
obtaining whole blood from a subject;
mounting one or more samples of said whole blood on one or more substrates;
exposing the samples to different levels or intensities of electromagnetic radiation;
detecting the level of genetic damage in each sample; and
comparing the levels of damage with predetermined values, or patterns of values.

2. A method as in claim 1 wherein the step of exposing samples to a different levels of electromagnetic radiation is carried out by altering the intensity of the electromagnetic radiation for each blood sample.

3. A method as in claim 2 wherein when the level of electromagnetic radiation is altered by altering the distance between the source of radiation

4. A method as in claim 1, wherein a first sample is exposed to a light intensity of ˜1.20 mW/cm2 for the second slide, a second sample to a light intensity of ˜0.80 mW/cm2, a third sample to a light intensity of ˜0.50 mW/cm2, and a fourth sample to a light intensity of ˜0.20 mW/cm2.

5. A method as in claim 1, wherein the plurality of samples may be taken as a single sample from a subject provided that it can be separated into a plurality of samples for testing.

6. A method as in claim 1, wherein samples are exposed to electromagnetic radiation for between 10 minutes and 30 minutes.

7. A method as in claim 1, wherein the samples are exposed to electromagnetic radiation for 15 minutes.

8. A method as in claim 1 wherein the step exposing the samples to varying levels of electromagnetic radiation is carried out varying the levels of electromagnetic radiation emitted.

9. A method as in claim 1, wherein no additional layer of barrier material is placed between the samples and the source of electromagnetic radiation.

10. A method as in claim 1 wherein no additional layer of barrier material is placed directly on top of the samples.

11. A method as in claim 1, wherein the whole blood comprises peripheral whole blood.

12. A method as in claim 1, wherein before exposing samples to different levels of electromagnetic radiation, the method includes the step of mounting the whole blood on a substrate.

13. A method as in claim 12 when the whole blood is mounted in mounting medium comprising 0.5% low-melting point agarose (<40° C.).

14. A method as in claim 1, wherein the EM radiation is UV radiation.

15. A method as in claim 1, which is used to screen subjects for a predisposition to skin cancer, lung cancer, breast cancer, bowel cancer, prostate cancer or colo-rectal cancer.

16. A method as in claim 1, which is used to screen subjects for not having a cancer, having precancerous cells, or having cancer where the cancers are including but not limited to skin cancer, lung cancer, breast cancer, bowel cancer, prostate cancer, colo-rectal cancer

17. A method as in claim 1, which is used to screen subjects for a predisposition to COPD, asthma, emphysema or polyposis coli.

18. An assay product comprising:
a substrate onto which a whole blood sample may be mounted; and
an EM radiation source positioned such that the surface of the substrate on to which the whole blood sample is mounted is exposed to EM radiation, wherein the intensity of radiation to which the sample is exposed is variable.

19. An assay product as in claim 18 wherein the distance between the sample and the EM radiation source is variable.

20. An assay product as in claim 18, wherein no additional layer of barrier material is placed directly on top of the sample.

21. An assay product as in claim 18, wherein no additional layer of barrier material is placed directly on top of the samples.

22. An assay product as in claim 18, wherein the whole blood comprises peripheral whole blood.

23. An assay product as in claim 18, wherein the whole blood is mounted in mounting medium comprising 0.5% low-melting point agarose (<40° C.).

24. An assay product as in claim 18, wherein the EM radiation is UV radiation.
